(19) United States
(12) Patent Application Publication
(10) Pub. No.: US 2014/0194802 A1
(43) Pub. Date: Jul. 10, 2014

(54) Protective Bandage Device

(71) Applicant: Todd P. Check, Indianapolis, IN (US)

(72) Inventor: Todd P. Check, Indianapolis, IN (US)

(21) Appl. No.: 14/147,514

(22) Filed: Jan. 4, 2014

Related U.S. Application Data
(60) Provisional application No. 61/749,512, filed on Jan. 7, 2013.

(51) Int. Cl. A61F 13/02

(52) U.S. Cl. CPC A61F 13/023
USPC 602/43

(57) ABSTRACT

The present invention relates to a protective bandage device and to methods of use thereof.

### FIELD OF THE INVENTION

The present invention relates generally to a protective bandage for use by a person in need thereof wherein said bandage covers a wound or ancillary medical device, and to methods of use thereof. More particularly, the protective bandage of the invention is placed on a person to form a protective shield over and around a wound or ancillary medical device.

### BACKGROUND OF THE INVENTION

A number of conventional first-aid bandages exist including, for example, contour bandages, adhesive bandages, surgical bandages, and gauze bandages that are applied evenly and smoothly over flat, contoured, and/or flexing areas of the body such as elbows, knees, heels, torso, legs, arms, etc. See, for example, U.S. Pat. Nos. D611,156, 7,605,299, and 7,176,343. These types of bandages are generally designed to provide self-adhesive tabs plus a centrally located absorbent pad. An adhesive backing is provided generally consisting of a flexible, breathable, gas-permeable, hydrophobic material. The adhesive backing is typically a woven or nonwoven plastic, paper, or other suitably soft material.

Bandages of this type do not protect a wound from contact by forces and/or objects in the environment. For example, persons who play sports not infrequently sustain injuries during the activity. Generally, a bleeding wound is cleansed, a contour bandage or a general-purpose bandage may be applied to the wound. Alternatively, the wound may go unbandaged and left exposed to the environment. Whether a bandage is applied or not, a person with such a wound may encounter disturbing and/or painful contact to the wound from the every day environment. For example, when a person sleeps and rolls over onto the wound, pressure may be felt on the wound sometimes causing pain. Additionally, as normal activity resumes, the person may accidentally hump the healing wound against an object thereby causing additional pain. Moreover, during the healing process, a wound will often seep blood or other exudate. If a conventional bandage is applied to a wound, blood or other liquid is absorbed by the absorbent pad of the bandage. Often an absorbent pad sticks to the wound causing additional pain when the bandage is removed. If no bandage is applied to the wound, thereby leaving the wound open, then blood seeping out of the wound will cause the wound to stick to clothing or bed sheets as the blood dries.

Thus, there remains a need for improved protective bandages for use in chronic and acute wound care.

### SUMMARY OF THE INVENTION

This summary is provided merely to introduce certain concepts and is not intended to identity any key or essential features of the claimed subject matter.

Certain variations of the invention provide improved bandage devices for medical use including but not limited to wound care, wound protection, and protection or shielding of ancillary medical devices attached to a patient.

Accordingly, it is an object of the present invention to provide a medically useful protective bandage device for use by a medical professional, patient, or any other individual in need thereof.

Another object of the present invention is to provide methods for facilitating wound healing.

Another object of the present invention is to provide methods for facilitating medical or veterinary care or treatment that utilizes ancillary medical devices including but not limited to tubes, catheters, stents, syringes, picc lines, and splints.

### BRIEF INSCRIPTION OF THE DRAWINGS

FIG. 1a is a perspective view of a first embodiment of a dome-shaped protective bandage device.

FIG. 1b is a side view of the embodiment shown in FIG. 1a.

FIG. 1c is a bottom view of the embodiment depicted in FIG. 1a.

FIG. 1d is a side cut-away view of a variation on the dome-shaped bandage device depicted in FIG. 1a.

FIG. 1e is a top view of the variation depicted in FIG. 1d.

FIG. 1f is a perspective view of as embodiment of a device of the invention placed on the arm of a patient and covering a PICC line.

FIG. 1g provides a top perspective view of an embodiment of a protective device of the invention when no compressive force is applied.

FIG. 1h provides a top perspective view of the bandage of FIG. 1g in which a compressive force is applied to each side of the bandage, illustrating flexible deformation of the bandage.

FIG. 2 is a side view of a deep, bowl-shaped embodiment of a protective bandage device.

FIG. 3a is a perspective view of a rectangular arch-shaped third embodiment of a protective bandage device.

FIG. 3b is a partial cross-sectional view of one embodiment of a male-female junction between two rectangular arch-shaped bandage devices.

FIG. 4a provides a side view of an embodiment of the invention showing the closed position of the device.

FIG. 4b shows the embodiment of FIG. 4a in an opening position.

FIG. 4c shows a top view of the embodiment of FIG. 4a.

FIG. 4d shows a top view of an embodiment of a comfort band of a device of FIG. 4a.

FIG. 4e shows a device of FIG. 4a including a tube passage.

### DESCRIPTION OF THE SELECTED EMBODIMENTS

As used herein the term “ancillary medical device” refers to any medical apparatus or device that is incorporated, applied, attached, or inserted onto or within the body of a patient for therapeutic purposes including but not limited to treating any chronic or acute disease or condition. Exemplary but non exclusive ancillary medical devices include catheters, PICC lines, stents, hypodermic syringe needles, tubes, field drainage tubes, feeding tubes, splints, etc.

As used herein, the term “sloping” or “sloped” refers generally to an aspect of the configuration of a wall of a bandage device of the present invention wherein the angle between the surface of the wall at or near the outer edge of the device is other than perpendicular; generally and preferably the sloped angle is less than 90°.

As used herein the term “wound” is broadly intended to include any insult injury, or condition of the body that may or may not cause pain, bleeding, tissue damage, tissue degeneration, or malfunction, whether intentionally inflicted for medial reasons, e.g. during surgery, or having arisen by accident, trauma, violence, or during sports activities, or as a result of disease, including but not limited to bruises, cuts, abrasions, scrapes, surgical incisions, ulcers, strains, surgical stitches, sprains, fractures, or degenerative diseases such as, for example, arthritis, inflammatory disease, fibromyalgia, lupus, cancer, etc, “Wound” may include healing, non-healing, and/or infected wound conditions potentially requiring professional medical intervention and/or hospitalization.

As used herein, the terms “person”, “patient”, and “individual” refer to a mammal including animals and humans on which a device of the present invention can be used.

As used herein, the term “rigid” or “substantially rigid” in reference to a device of the invention generally refers to a device of the invention. Rigidity, as used herein, does not exclude some degree of flexibility, for example, having the capacity for bending to better conform to an area of the body on which a device is applied.

The phrase “environmental insult” is used broadly herein to mean any force, contact, or impact such as a bump, scrape, bruise, etc. that may result in a painful, disruptive, or harmful contact with the body and/or with a wound on a patient.

In one aspect, a device according to the present invention is provided as a means to foster or promote healing of a wound when used alone or in combination with other medical treatments and/or devices including hot not limited to band-aids, bandages, gauze, or other medical treatments including hut not limited to ingestion or application of medicaments including orally or topically administered medicaments or pharmaceuticals sack as antiseptics, antifungal agents, antiviral agents, anti-inflammatory agents, pain relievers, etc.

A device of the invention provides a protective barrier or shield for a wound and/or an ancillary medical device attached to a patient against undesired environmental insults such as bumps, pressure or other undesired contact that can cause pain and/or interrupt the healing process. In the case of ancillary medical devices such contact may disrupt or dislodge such devices thereby causing pain and impeding the clinical benefit intended for such devices. A bandage device of the present invention can be used with or without absorbent pads and other dressings depending on the particular need of the patient.

In one aspect, a device of the invention can be used in conjunction with ancillary medical devices including but not limited to peripherally inserted central catheter devices i.e. “PICC lines” to shield such devices, for example, to prevent dislodgement or displacement from the body. As such, a device according to the present invention promotes healing and avoids additional discomfort or pain that can be caused by undesired environmental insults. In addition, the healing process is allowed to proceed without interruption as, for example, when an incipient scab is undesirably dislodged by a disruptive force encountered in the environment.

For purposes of promoting an understanding of the principles of the invention, reference will now be made to certain aspects of embodiments of the invention as illustrated in the drawings and disclosure herein. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

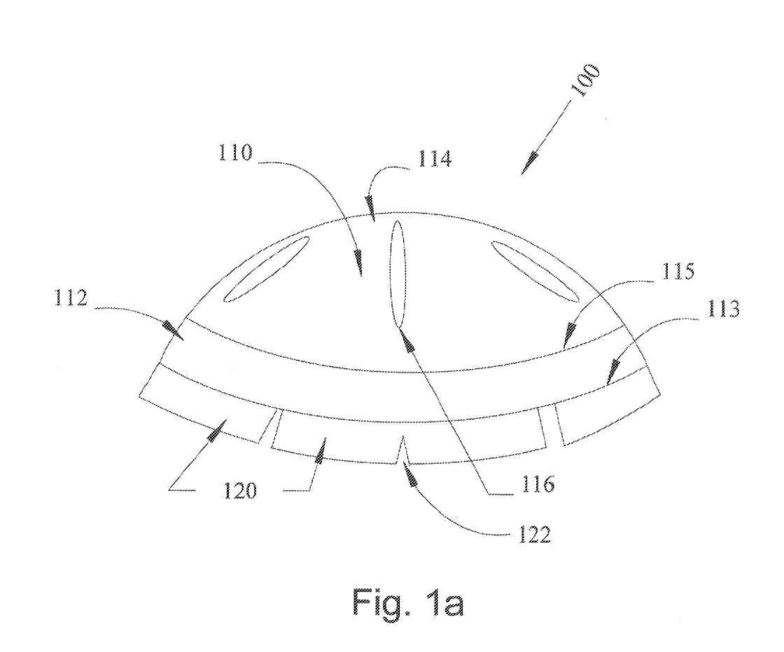

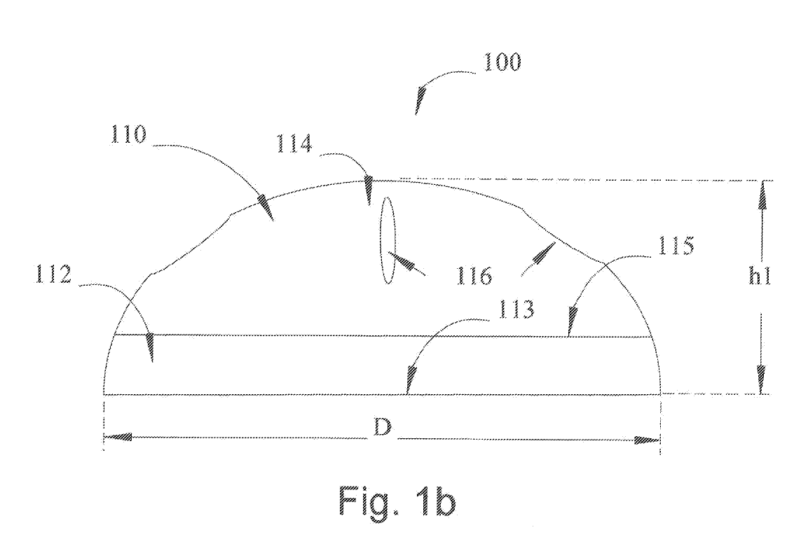

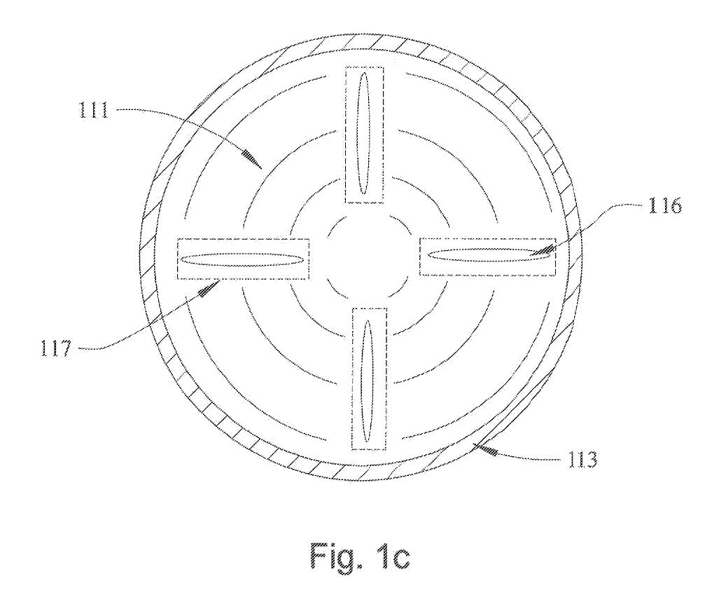

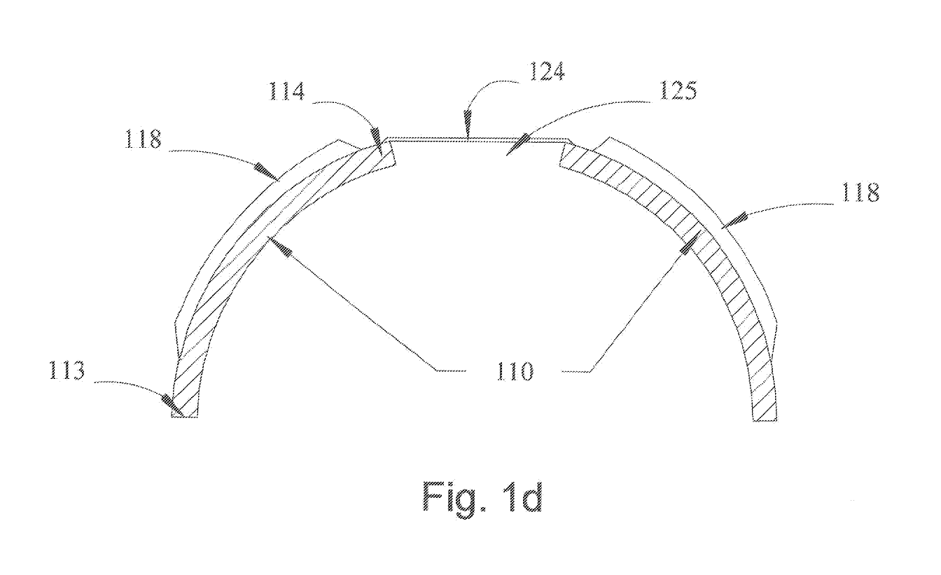

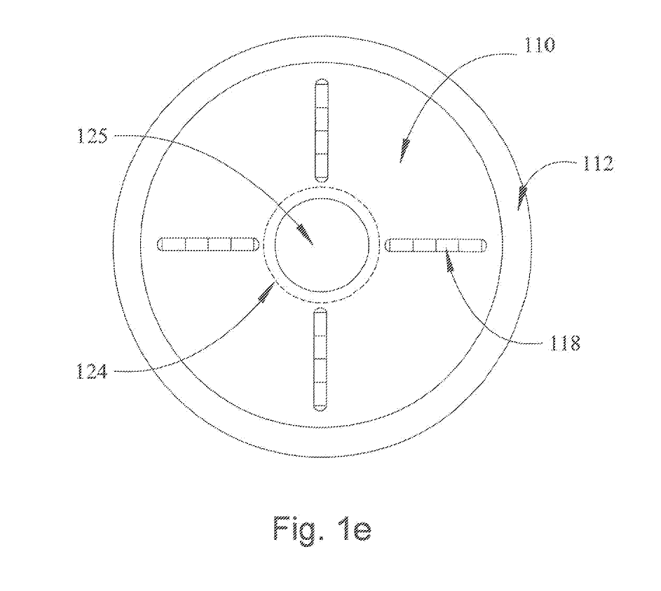

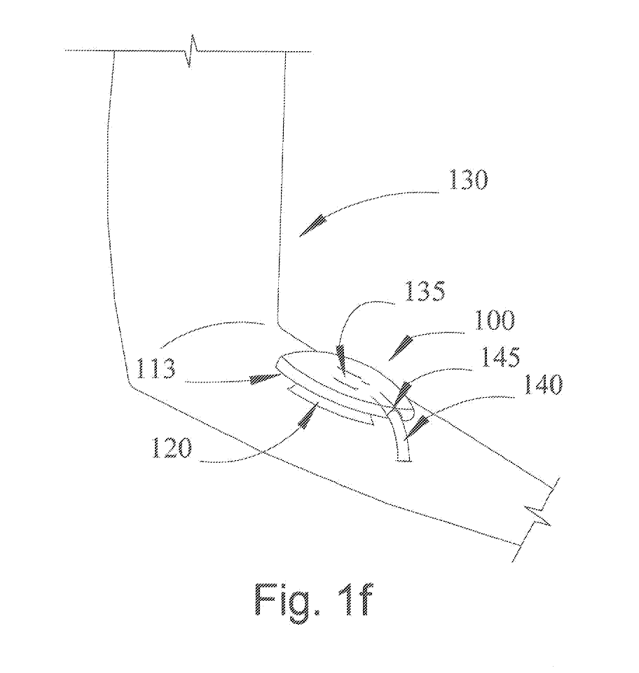

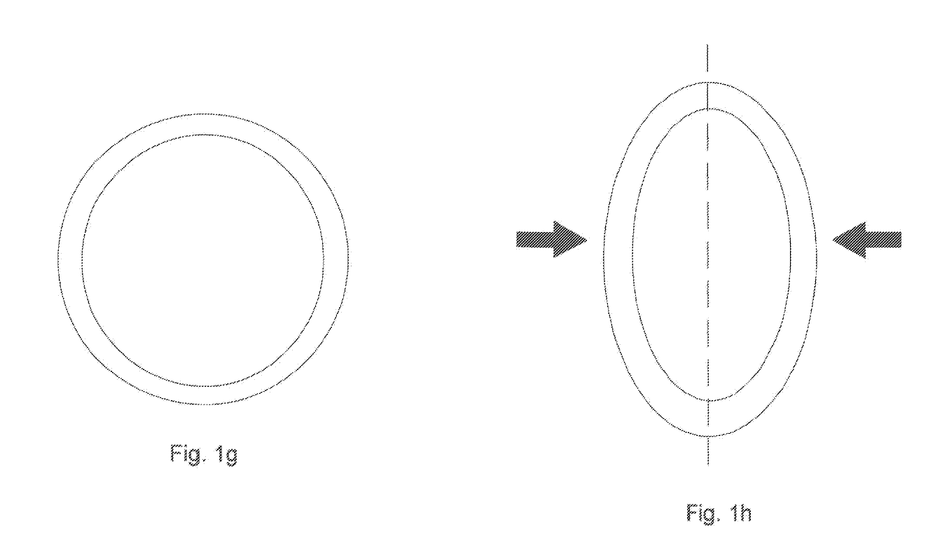

FIGS. 1a-1h illustrate various embodiments of a dome-shaped protective bandage device of the invention. As depleted in FIG. 1a, in one embodiment the bandage 100 includes a wall 110 that extends radially from a central apex portion 114 to a comfort baud 112. In the illustrated embodiment, the wall 110 forms a dome shape with a central apex portion 114 that extends to an outer perimeter 113. The outer perimeter 113 and central apex portion 114 define a height dimension h1 (See FIG. 1b). The height h1 can be of any desired dimension but generally ranges from about 0.25 inches to about 5 inches; alternatively from about 0.5 inches to about 3 inches. The wall 110 has a generally hemispherical or semispherical shape. As illustrated in FIG. 1c, the hemispherical shape defines a concave interior portion 111. Other shapes of wall 110 can include circular, polygonal, or an elliptical shape. Preferably, the wall 110 defines a sloping surface that is preferably not perpendicular at the junction with the outer perimeter 113. That is, preferably the angle at the junction between the wall 110 and fee outer perimeter 113 is less than 90%, for example from about 10° to about 80°; preferably from about 20° to about 50°. The circular dome shape for wall 110 is beneficial in that as a force is applied to the bandage 100, the force is dissipated in all directions. Moreover, the dome shape of wall 110, while accommodating any part of the body, is also useful in covering a wound on curved body parts such as, an elbow, shoulder, knee, ankle, and heel, to name a few. The wall 110 is configured to cover and protect a wound without contacting the wound itself. One embodiment of the invention includes a comfort band 112 that attaches to the wall 110, or is integral therewith around the outer edge portion 115. The comfort band 112 may extend away from the outer edge portion 115 and define the outer perimeter 113 of the device for contacting the skin surrounding a wound. In general, wall 110 forms a bubble or shield of protection over a wound. Moreover, wall 110 can facilitate keeping the wound in a clean, and if desired, sterile environment. Sterility of a device of the invention can be provided by any suitable method known to the skilled artisan including physical and chemical treatments, for example, treatment of a device with chlorohexidine, and/or by including a chemical sterilizer on the device or part thereof, for example, by providing chlorohexidine around the edge of a device, or on the comfort band or other part of the device.

In one embodiment, wall 110 of the dome-shaped bandage provides a substantially continuous surface. In another embodiment of the dome-shaped bandage, wall 110 provides at least one hole or vent of any desired size and/or shape to facilitate air circulation during use of the device by an individual in need thereof. As illustrated in FIG. 1c, the holes or vents 116 may be covered with a protective material or membrane 117 that allows for air flow while providing a barrier to entry of dust or other environmental debris. Any suitable material can be used for the protective material 117 including natural or synthetic fiber material or mesh material such as cotton or other plant-fiber material, and any suitably porous or breathable plastic material.

The wall 110 can have different sizes to comfortably fit over any desired area of the body to which the bandage 100 is applied, and/or to accommodate ancillary treatment modalities such as absorbent pads and/or ancillary medical devices which may require additional space under the dome. For example, as illustrated in FIG. 1b, wall 110 has a diameter D which can range from about 0.5 inches to about 8 inches. Preferably, diameter D is from about 2 inches to about 4 inches. The dome-shaped embodiment can also have varying height dimension h1 (FIG. 1b); preferably h1 is from about 0.5 inches to about 5 or more inches; in one aspect, h1 is less than or equal to one-half the diameter D. For some applications, it is desirable that the height of a device of the invention provide adequate space for packing a wound with bandages, dressings, and/or medicaments to facilitate healing of wounds including chronic non-healing wounds.

Wall 110 can be made of various materials preferably materials that provide some degree of hardness or rigidity but preferably with some flexibility retained. In one form, wall 110 is made of any suitable material known to the skilled artisan that provides sufficient rigidity to withstand a force or pressure without collapsing onto a wound. Suitable but non-exclusive materials for wall 110 include various synthetic or semi-synthetic plastics or thermoplastic materials including but not limited to polystyrene, PVC, nylon, polycarbonate, and acrylic that can be clear, colored, opaque. For example, a wall 110 and/or other part of a device of the invention can be manufactured from an opaque, colored, translucent or transparent plastic.

In the illustrated embodiment of FIGS. 1a-1c, wall 110 has a substantially smooth exterior surface; however, in other embodiments, wall 110 can include one or more ridge structures 118 to strengthen wall 110 (See FIGS. 1d and 1e). FIGS. 1d and 1e depict a-bandage having four ridges 118 spaced at about 90° intervals and distributed substantially in a radial configuration around the central apex portion 114. It should be understood that other configurations and arrangements for ridges are within the scope of the invention including bandage devices having from 2 to 8 ridges spaced approximately substantially evenly or unevenly and being spaced at about 45° to about 180° intervals around wall 110 substantially in a radial or non-radial configuration. In some embodiments, wall 110 can include pictures or illustrations tor decoration or entertainment, for example, when the device is to be used by children.

The embodiments of the invention may include a comfort band that may be integral with the wall portion or top portion of the device. Alternatively, a comfort band may comprise a separate member that may or may not be detachable from the rest of the device. In the illustrated embodiment of FIG. 1a, comfort band 112 forms a ring or circular shaped structure around the outer perimeter of wall 110. Comfort band 112 may be a separate member or, alternatively, a contiguous portion of wall 110. In the latter case, the comfort band could be formed, for example, by turning the wall edge under, or by smoothing the edge portion such that any sharp edges around the outer perimeter are removed. In other embodiments, comfort band 112 can have a different configuration or other shapes such as oval, elliptical, polygonal, or any other configuration to engage the wall 110 as desired. Comfort band 112 can be made of the same material as wall 110 or of a different material. Comfort band 112 can have the same or different material properties as wall 110. For example, wall 110 can be fabricated from a plastic material that provides rigidity while comfort band 112 can be fabricated from a more pliable material such as a softer plastic or fabric. Desirably, comfort band 112 feels soft on a persons skin around a wound. Comfort band 112 generally is sized larger than a wound so as not to contact or rest against the wound.

In the illustrated embodiment of FIGS. 1a and 1b, the wall 110 includes four vents 116 wherein each of vents 116 is spaced about 90° (90 degrees) from an adjacent vent substantially radially around the central portion 114. In other embodiments, wall 110 can include a greater or lesser number of vents 116 or none at all, and the vent(s) can be configured in any manner, for example, radially or non-radially. The vents 116 are beneficial in allowing air flow and moisture escape from the wound protective bandage 100 when the bandage 100 is applied over a wound or ancillary medical device. As shown in FIG. 1c, in a preferred embodiment the vents 116 are covered by a covering material or membrane 117 that allows free air flow while blocking entry of environmental debris, dust, or other material. The covering material 117 may be placed over the vests 116 on the top side of the dome, or, as illustrated in FIG. 1c, on the underside of the dome. While FIG. 1c shows the covering material 117 to be approximately the same size and shape as the vents 116, other configurations and/or shapes can be used. For example, the covering material 117 could comprise a single piece of material that covered one or a plurality, including all, vents 116.

In another variation of a bandage of the invention, as illustrated in FIGS. 1d and 1e, the wall 110 includes a central aperture 125 at the central apex portion 114. The central aperture 125 can be of any suitable and/or desired size, shape, and dimension to facilitate access to a wound or ancillary medical device without having to dislodge and remove the bandage from the patient, for example, when applying a medication to a wound or when checking or servicing an ancillary medical device. In a preferred embodiment, central aperture 125 has a diameter of from about 0.5 inches to about 3 inches and is covered by a protective membrane 124 that can be soft and/or pliable. The protective membrane 124 can be lifted as needed to expose and provide easy access to a wound or ancillary medical device without disturbing the bandage. Alternatively, central aperture 125 can have a hard and/or rigid coveting or closure means that may be opened or closed for easy access to a wound. Alternatively still, central aperture 125 can be left open without any covering. The protective membrane can be made from any suitable material including but not limited to natural or synthetic fiber mesh material, gauze, plastic, thermoplastic, etc. Preferably, the membrane 124 has an adhesive applied, for example, around the edge of membrane 124 to facilitate repeated lifting and replacing the membrane onto the bandage as desired.

The bandage 100 also preferably includes attachment means by which to secure the device to a patient. Any suitable attachment means can be used including, but not limited to, adhesives that are applied to the skin, adhesive strips, tape, wraps, etc. FIG. 1a illustrates one embodiment in which adhesive tabs 120 are connected to a portion of the wall 110 and/or the comfort band 112 wherein the adhesive tabs 120 are configured to attach to the skin surrounding a wound or ancillary medical device until it is desired to remove the tabs 120 (FIG. 1a). Each of the adhesive tabs 120 is spaced around the central portion 114 at about 90° from an adjacent adhesive tab 120. Other embodiments can include a greater or lesser number of adhesive tabs 120 wherein the adhesive tabs 120 would be spaced around the central portion 114 in a spaced-apart relation to the wound. In yet another embodiment, a continuous adhesive tab can be applied along the comfort band 112 or outer edge 113. In the illustrated embodiment, the adhesive tabs 120 have a generally rectangular shape and are sized to fit around the bandage. In other embodiments, the adhesive tabs 120 can also have another shape and/or size. The adhesive tabs 120 cart have a different size and/or shape from one another. Additionally, as illustrated in FIG. 1a, each of the adhesive tabs 120 may include a relief slit 122 that facilitates the adhesive tab 120 to lay flat against a person's skin when it is applied. In other embodiments, an adhesive tab 120 is continuous, that is without the relief slit 122.

In another variation of the dome-shaped embodiment, one or more ancillary medical device passages or holes are located on the top portion for fire accommodation of ancillary medical devices. In a preferred embodiment, the one or more passages are located near or around the radial edge. Such passage(s) are particularly useful in a hospital or institutional environment when the device is used to protect an ancillary medical device that may include tube(s) that emerge from the site of placement. The ancillary medical device passages of this type provide an outlet for such tubes that avoids disturbing the secure placement of the device on the patient. FIG. 1f illustrates placement of a dome-shaped device 100 on the arm 130 of a patient. As illustrated, the device 100 is placed over an ancillary medical device 135 such as a picc line from which the tube 140 passes through an ancillary medical device passage hole 145 located at the outer edge 113 of the device.

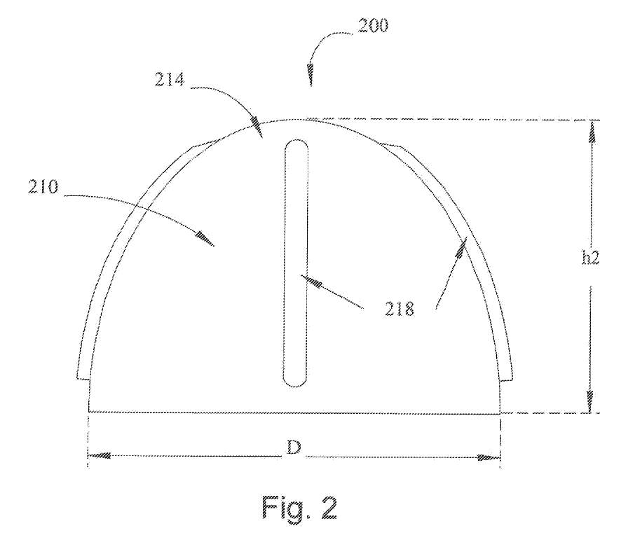

FIG. 2 illustrates a second embodiment of a wound protective bandage 200 according to the present invention. Bandage 200 is similar to bandage 100; therefore, for the sake of brevity similar features will not be extensively described. Bandage 200 also includes a wall 210. Wall 210 forms a bowl shape with a central portion 214. In the illustrated embodiment, wall 210 includes a plurality of ridges 218 to strengthen the wall 210. In other embodiments, wall 210 includes a single ridge or is smooth and lacks ridges. As with the first embodiment, the second embodiment may also include one or more vents to facilitate air flow and/or a central aperture. Wail 210 can assume any size to fit over a wound but not contact or touch the wound. For example, the embodiment of FIG. 2 can have a diameter D of from about 1 inch to about 6 inches. The configuration, shape, and size of wall 210 may be beneficial for positioning bandage 200 over a wound or ancillary medical device on a part of the body that is not flat or smooth, for example, a knee or elbow. As with the first embodiment (FIGS. 1a-1c), bandage 200 includes a height dimension h2. Preferably, h2 is greater than the height dimension h1 of the first embodiment. Height dimension h2 can range from about 1 inch to about 8 inches; preferably, h2 is from about 2 inches to about 5 inches. In one aspect, the embodiment of FIG. 2 provides expanded space under the dome or top portion that can be useful for packing wounds with gauze or other dressing and/or for providing a better fit over certain regions of the body.

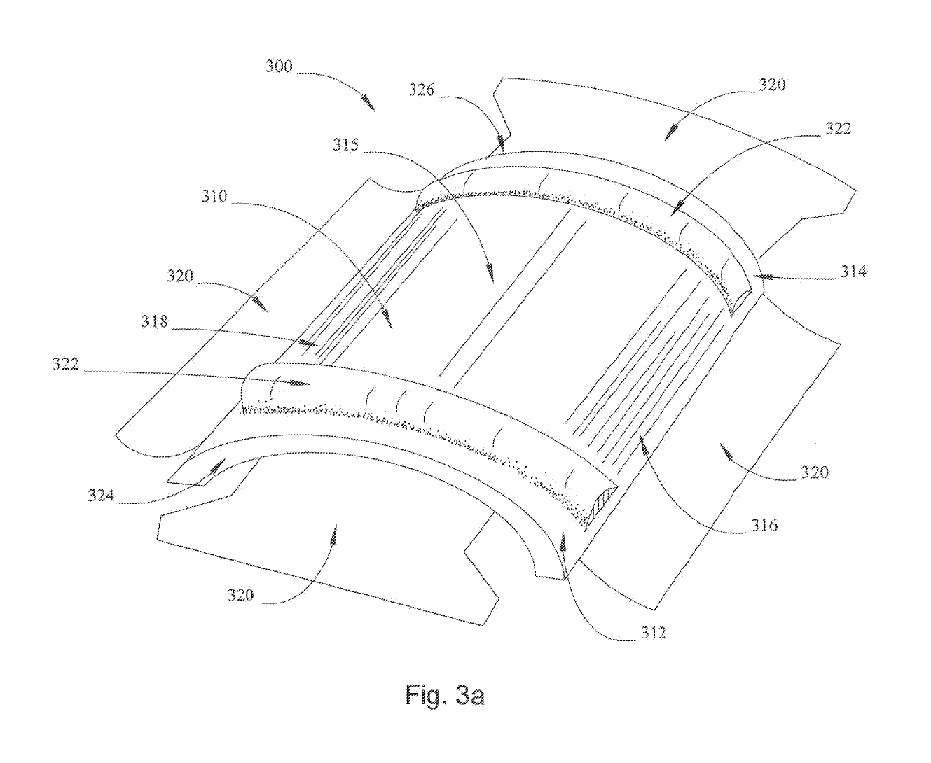

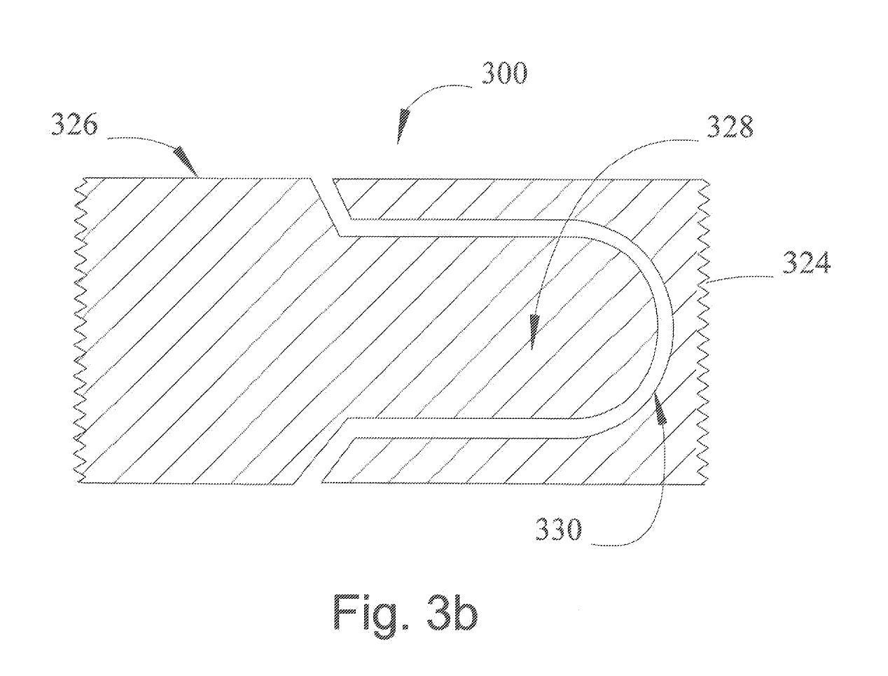

FIGS. 3a and 3b illustrate aspects of a third embodiment of a wound protective bandage of the invention. Bandage 300 includes a wall 310 that extends between a front portion 312 and an opposite a rear portion 314 with a central portion 315 spanning between the front portion 312 and the rear portion 314. The device of the third embodiment includes a front edge portion 324 and a corresponding rear edge portion 326. The wall 310 also includes a right side portion 316 and a left side portion 318. The wall 310 is similar to wall 110; however, wall 310 is configured differently. Wall 310 is similar to wall 110 in that wall 310 is configured to cover or bridge over a wound or ancillary medical device without contacting or touching same. In the illustrated embodiment, wall 310 has a substantially rectangular arch shape. Therefore, when bandage 300 is placed over a wound or ancillary medical device, the right side portion 316 and the left side portion 318 contact the skin near the wound. However, the front portion 312, rear portion 314, and central portion 315 should not contact the wound or ancillary medical device if the bandage 300 is sized and positioned properly on the user. As such, wall 310 can be manufactured in any range of sizes to fit the desired body area and wound to which the bandage 300 is applied. For example, in the illustrated embodiment, wall 310 has a length of about 3 to 4 inches and a width of about 2½ inches. Wall 310 generally has an arch shape but can be other than rectangular such as, for example, square or other polygonal arched shape. Similar to bandage 100, bandage 300 can also be used to cover or bridge over an ancillary medical device, for example, a peripherally inserted central catheter, an intravenous catheter, stitches, splint, stent, syringe, or other medical device inserted into or onto the body wherein bandage 300 is applied at the point of entry or attachment to the body.

The front portion 312 and the rear portion 314 can be configured in a variety of ways to enable a reversible or non-reversible coupling between two or more bandages. In a preferred embodiment the configuration enables a reversible coupling of a plurality of bandages. In this aspect, the embodiments include any suitable means known to the skilled artisan for reversibly coupling mechanical parts, in particular means to couple or connect bandages of the present invention. Suitable coupling means include, but are not limited to, interlocking means such as male-female type connections, adhesive couplings, and connections using Velcro. In one embodiment, the front portion 312 and rear portion 312 can be configured to form an interlocking mechanism with another bandage having corresponding mating front and rear portions 312 and 314. For example, as shown in FIG. 3a, a first bandage 300 has at the front portion 312 and rear portion 314 connecting ridge-like structures 323 that enable reversibly connecting two or more bandages 300 in an end-to-end fashion by providing male and female-type structures to enable a reversible connection. In one embodiment, as depleted in FIG. 3a, connecting ridge-like or ridge-shaped structures 322 are configured to have an outer convex surface and a hollow concave interior region. The concave interior region is configured to receive the outer convex surface of another bandage 300 thereby coupling two bandages together. For example, the front portion 312 of one bandage comprises a female receiving end and the rear portion 314 of a second bandage 300 comprises a male end that is inserted into the front portion 312 of the first bandage 300 to form a reversibly interlocked pair between the first and second bandages 300. Any desired number of bandages 300 can be attached together to form a continuous line of bandages 300 to cover larger wounds or one or more ancillary medical devices.

Wall 310 can be made of various materials that are preferably hard or rigid after formation of a desired shape. In one form, wall 310 is made of any material that is sturdy enough to withstand constant pressure without collapsing onto the wound. Moreover, wall 310 is preferably sturdy or rigid enough that when pressure is applied to the wall 310, none of the front portion 312, rear portion 314, central portion 315, right side portion 316, or lift side portion 318 will deflect to contact the wound. One example material for wall 310 includes any suitable plastic or thermoplastic material that can be clear, colored, or opaque. As with the dome-shaped embodiment, in some embodiments of an arch-shaped device, wall 310 includes one or a plurality of structural stability ridges to enhance the strength of the bandage. In other embodiments, wall 310 has a substantially smooth surface (See FIG. 3a). Moreover, wall 310 can include pictures or illustrations for decoration.

The bandage 300 also includes one or more adhesive tabs 320. FIG. 3a depicts a device with four adhesive tabs 320 wherein one of tabs 320 is connected to each of the front portion 312, rear portion 314, right side portion 316, and left side portion 318. Adhesive tabs 320 are similar to adhesive tabs 120 and are configured to attach to the skin surrounding a wound or medical device until it is desired to remove the bandage 300. In the illustrated embodiment, the adhesive tabs 320 have a generally rectangular or polygonal shape. The adhesive tabs 320 can be of any suitable size sufficient to securely attach a bandage 300 to a patient. For example, adhesive tabs 320 can range in she from about 0.5 inches to about 5 inches in length, and from about 0.5 inches to about 2 to 3 inches in width. In other embodiments, the adhesive tabs 320 can have another shape and/or size. Moreover, the adhesive tabs 320 on one bandage 300 can have a different size and/or shape from one another.

FIG. 3b depicts another embodiment for bandage 300 showing a partial cross-sectional view of the rear edge 326 and front edge portions 324 of two interlocking bandages 300 to illustrate another means for reversible connecting two or more bandages 300. In this alternative embodiment, the interlocking ridges 322 of the embodiment of FIG. 3a are replaced by interlocking male-female structures at the edges of bandage 300. A rear edge portion 326 of one bandage 300 has a male structure 328 that interlocks with the front edge portion 324 of another bandage 300 having a female structure 330.

A device of the present invention can be provided in unit or multiple-unit package form, preferably in sterile form. Sterilization of a device of the invention can be provided using any method known to the skilled artisan including but not limited to chemical and physical means such as radiation, heat treatment, and treatment with gases such as ethylene oxide, and with liquids such as alcohols, aldehydes, phenolics, etc. For some uses, it may be desirable to infuse a portion or all of a bandage device of the invention with an antiseptic agent such as chlorohexidine, for example, around the outer edge of the device or portion thereof that comes in contact with the skin.

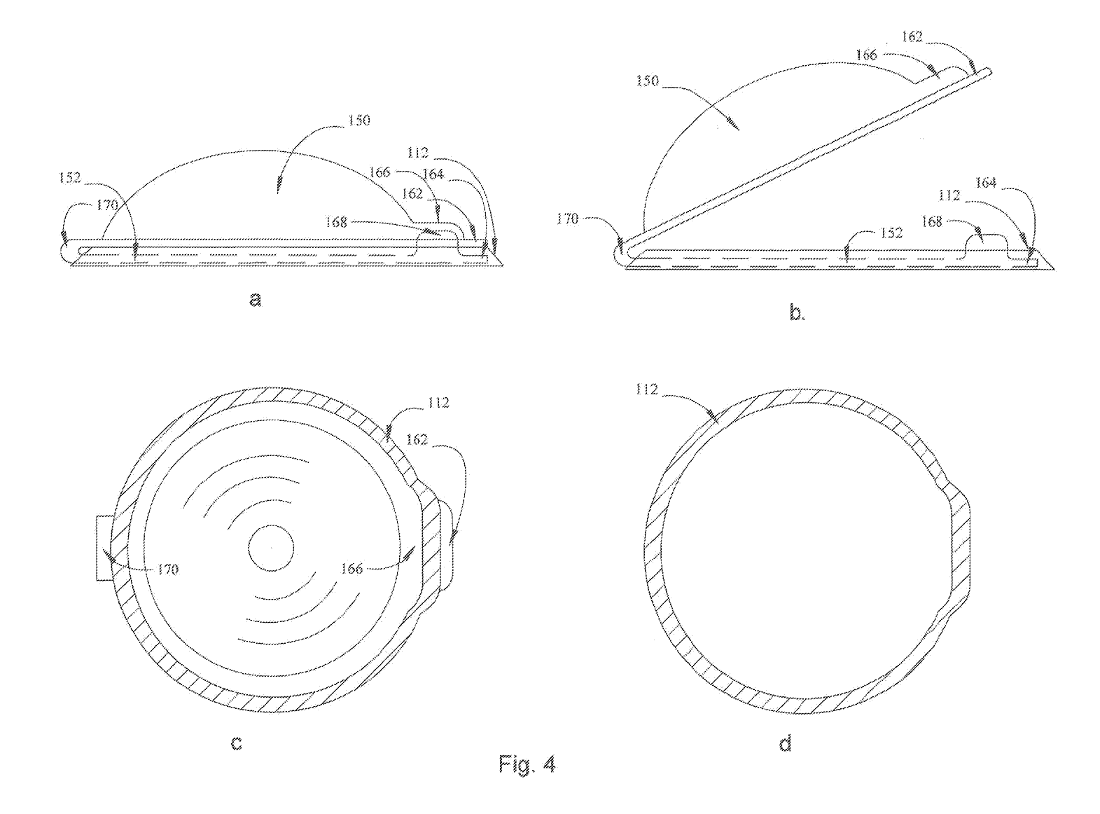

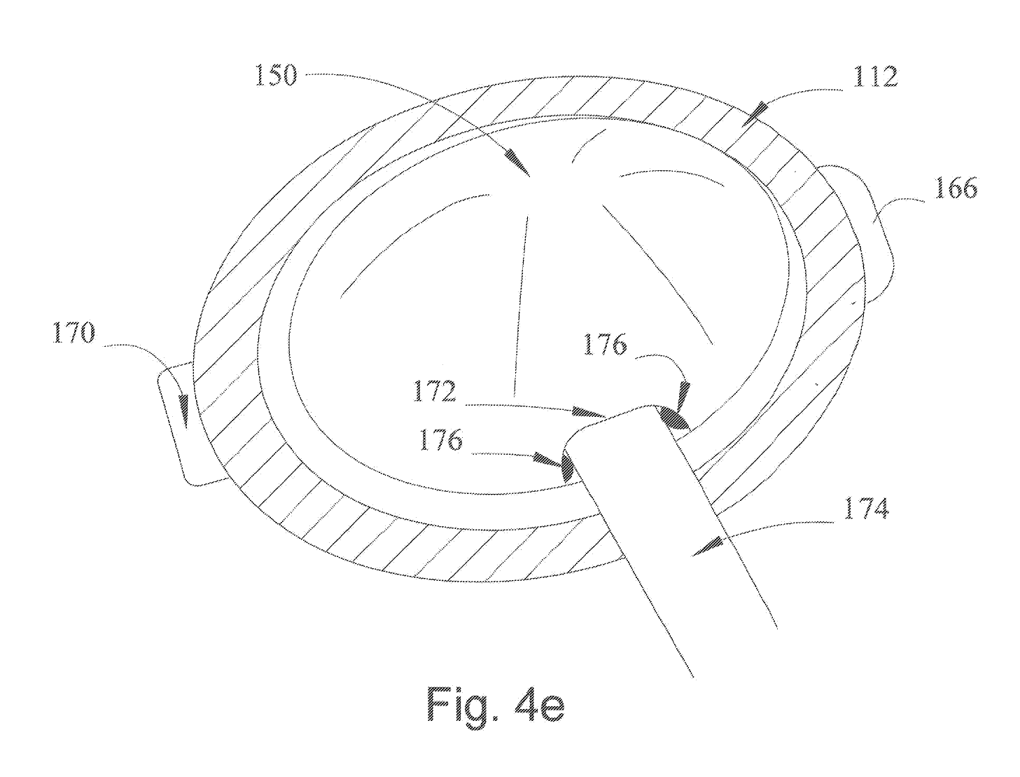

For added convenience and/or utility during use of a bandage of the present invention, it may be advantageous or desirable to provide easy access to a wound without having to remove the entire bandage after attachment to a patient. For example, it may be desirable for purposes of applying medications to a wound or for cleaning a wound or for packing a wound to have easy and open access to the wound without having to dislodge and remove the entire bandage. For this, purpose, and in another embodiment of the invention, as depicted in FIGS. 4a-4e, a bandage device of the present invention provides a top portion 150 and a bottom portion 152. In one embodiment of this aspect, the bottom portion 152 includes a detachable comfort band 112 that partially or completely surrounds the bottom portion for contacting the skin around a wound during placement of the bandage over a wound. The comfort band 112 may also be integral with the top portion 150 or bottom portion 152, for example, the edge of said top portion or bottom portion can be smoothed to be comfortable on the skin.

The comfort band 112 may or may not include an adhesive on the skin-contact side of the comfort band for ease of attachment of a bandage to the skin of a patient without the need for additional attachment means such as, for example, strips of tape applied to the bandage device for attachment to the skin. In one aspect of this embodiment, the top portion 150 and bottom portion 152 operatively couple to provide an open position, as depicted in FIG. 4b, in which the top portion 150 is pulled away from the bottom portion 152 to expose the wound area (See FIGS. 4a-4b). In one embodiment of this aspect of the invention, the top portion 150 and bottom portion 152 are configured with coupling members 166 and 168 respectively that operably-couple to allow the top and bottom portions to reversibly couple. For example, as depicted in FIGS. 4a-4c, the top portion and bottom portion may include coupling members at or near the perimeter of said portions. The embodiment depicted in FIG. 4 further provides a hinge member 170 which may be a separate, discrete member, or alternatively, as depicted in FIGS. 4a-4c, an integral member with said top and bottom portions. The hinge member 170 enables opening and closing the bandage without completely separating the top and bottom portions.

For ease of opening a bandage of this embodiment, the top portion and/or bottom portion may include a tongue or tab member that provides a convenient means for grasping during opening and closing the bandage. The tab member can be of any shape including square, oval, circular, rectangular, or any other shape that can be easily grasped for opening and/or closing the device. In one embodiment of this aspect, as depicted in FIGS. 4a-4b, a top portion tab 162 and a bottom portion tab 164 are approximately the same size and shape. Alternatively, either the top portion tab 162 or bottom tab 164 may be larger than the other, or of a different shape than the other to facilitate ease of grasping doing opening and closing.

The top portion and bottom portions may be configured in any manner to allow reversible coupling using any suitable means known to the skilled artisan, for example, snap-closure means, Velcro, tape, tongue and groove joint, ball and socket joint, etc. In one embodiment, the top and bottom portions remain conveniently attached while in the open position, for example, by means of a joining hinge that allows the top portion to rotate away from, or pivot away from the bottom portion without completely detaching the top portion from the button portion. In another embodiment, the top and bottom portions completely detach from one another in the open position.

The top portion, or top and bottom portions, may include any one or more other features as described herein for other embodiments of the invention including, but not limited to, one or more vents for enhanced air flow, and one or more ridge structures to enhance the stability of the device against forces from the environment. The top portion may also include a central aperture.

The top and bottom portions may have any desired size or shape including, but not limited to, round, oval, hemispherical, rectangular, square, triangular, polygonal etc. as described for other embodiments.

In another aspect, as depicted in FIG. 4e, the top portion provides a tube passage 172 for accommodating a tube 174, or other ancillary medical device such as a peripherally inserted central catheter, i.e. “PICC-line”. In one aspect of this embodiment, the tube passage 172 includes means to secure a tube of any size, or other device when positioned in the bandage device, i.e. stabilize the position of such a tube relative to the bandage device or top portion thereof. This feature is intended to reduce or eliminate movement of a tube once it is placed in the tube passage 172. For example, in one embodiment, a tube passage includes a securement device or securement means, for example, channel projections or knobs 176, which can be positioned anywhere in the tube passage 172, or portion thereof, for example around the side(s) and/or top of the tube passage, for making contact and creating frictional force against a tube wall inserted in the tube passage. The securement means, for example, knobs can be configured to accommodate tubes of various sizes, for example, by tapering. Other tube-securing means and configurations for stabilizing a tube inserted through the tube passage 172 would be readily apparent to the skilled artisan including, for example, Velcro, adhesives, tape, roughened surface texture of the tube passage, etc. Tube passage 172 and/or the securement means thereof, preferably incorporates an antiseptic agent, for example, chlorohexidine, for maintenance of sterility.

The top and bottom portions can be made of any of various suitable materials, preferably materials that provide rigidity after formation of a shape but with some flexibility retained to allow the bandage to comfortably conform to the surface of the skin or body part to which the device is attached. In one form, the top and bottom portions are made from any suitable material known to the skilled artisan that is sturdy enough to withstand ordinary environments pressures and/or forces without collapsing onto a wound. In a preferred embodiment, a bandage is not so rigid that it cannot bend or fold so as to conform with the contour of the surface of skin to which it is applied. For example, some regions of the body, for example the arm, may have a contoured shape that may not be adequately covered by a non-conformable, flat-surfaced bandage. Thus, in one aspect, a bandage of the invention provides some flexibility such that it can bend or conform to body shape where a bandage is applied. FIG. 1g depicts a bandage of the invention in the relaxed state, that is, in the absence of any compressive force. FIG. 1h shows the same bandage under a compressive, or squeezing, axial force, illustrating the desired feature of flexibility of the device.

Suitable but non-exclusive materials for a device of this embodiment of the invention include various synthetic or semi-synthetic plastics or thermoplastic materials including but not limited to polystyrene, PVC, nylon, polycarbonate, and acrylic that can be clear, colored, translucent, or opaque. As with other embodiments, the top portion and bottom portion may be manufactured from the same or different materials.

### Methods of Use

In another aspect, the present invention relates to methods of using a bandage device of the invention to promote healing of a wound, including, but not limited to, surgically-induced wounds, or traumatic wounds caused accidently, or wounds sustained during sporting activities. Use of a protective device of the present invention is expected to facilitate the healing process by reducing environmental insults such as bumps, scrapes, etc. that otherwise might retard the healing process and inflict additional pain and/or discomfort to a patient. A protective device of the present invention is also expected to provide clinical benefit to patients including but not limited to, those who are hospitalized or institutionalized where treatment may involve use of ancillary medical devices such as PICC lines, stents, tubes, splints, syringes, catheters, etc. Such ancillary medical devices generally protrude from a patient's body or skin, and are easily displaced or dislodged, impeding the purpose for which they are utilized and possibly inflicting additional pain to a patient.

One embodiment of this aspect of the invention relates to a method for treating a wound comprising attaching one or more bandage devices of the present invention to a patient in need thereof. In another embodiment of this aspect, the invention relates to a method for protecting or shielding one or more ancillary medical device(s) attached to a patient from disruption or dislodgement comprising covering said ancillary medical device(s) with one or more bandages of the present invention.

A device of the present invention can be used by medical and or veterinary professionals in or out of a hospital environment, or by non-medical professionals for in-home use. For use by medical professionals, including doctors, veterinarians, and nurses as well as other medical, care professionals, a device of the invention can be used for any type of wound care and/or protecting any type of ancillary medical device that may be used on a human or veterinary patient. For example, in a hospital or institutional environment, including nursing homes, a device of the invention can be attached to a patient to protect any type of vessel, tube, or device that is inserted into or otherwise applied onto a patient's body for any purpose including, but not limited to, nutrition, hygiene, and therapeutic purposes including PICC lines, feeding times, syringes, catheters, and the like (See, for example, FIG. 1f).

The methods according to this aspect of the invention can be applied alone or in combination with other treatments. In one aspect a bandage of the present invention is applied immediately after an individual sustains a wound or injury, or soon thereafter, or has an ancillary medical device attached with or without other treatments. For example, in one embodiment a bandage of the invention is attached to a wound without concurrent additional treatment(s) or treatment modes. In another embodiment, a bandage of the invention is applied to a wound, including a bleeding wound, that is concurrently or sequentially dressed or treated with other devices or modalities, for example, a band-aid, absorbent pad(s), gauze pad(s), splint, etc.

For some wound care applications it may be desirable to pack a wound, for example, with gauze or other suitable wound packing material. In such cases, a device of the invention can provide a domed region having sufficient space over the wound to accommodate such packs. In some aspects, a bandage of the invention is used in conjunction with other therapeutic or pharmaceutical treatments including topically applied or orally administered medications, such as, but not bruited to, antibiotics, antiseptics, anti-inflammatory agents, pain reducers, etc.

A bandage of the invention can be used for any type of wound care including, but not limited to, healing and non-healing wounds such as chronic wounds, diabetic ulcers, leg ulcers, pressure ulcers, stasis ulcers, surgical wounds, traumatic wounds, etc.

In another embodiment of this aspect of the invention, a bandage of the invention is used concurrently with, or in succession, or sequentially with other treatments. For example, a wound or ancillary medical device may be initially treated or managed by conventional means, for example, by application of a splint or absorbent pad to absorb blood flow, followed thereafter by application of a protective bandage of the present invention. For example, on day 0, a fracture or bleeding injury is sustained by a patient and a split or absorbent pad is applied as appropriate to the injury. A device of the present invention can be applied in conjunction with such other treatments, for example, on day 0, day 1, day 2, day 3, day 4, day 5, day 6, day 7, day 8, day 9, day 10 or any time thereafter the injury is sustained, as deemed appropriate by the patient or care-giver. In certain cases, it may be advantageous to apply a protective bandage of the present invention on day 1 or later after the injury has occurred, for example, to allow for the control of blood flow.

In some embodiments, bandages are applied to a patient by attaching the bandage to the appropriate site on the patient using adhesive strips or any other means for attachment including but not limited to tape, gauze wrappings, etc. Bandage devices having a centrally located aperture in the wall, or one in which the top and bottom portions reversibly couple are especially convenient in cases in which a wound must be dressed during the healing process to allow access to the wound without having to remove the bandage device from the patient which otherwise may cause pain and/or disruption to the healing process.

While the invention has been illustrated and described in detail in the drawings and foregoing description, the same is to be considered as illustrative and not restrictive in character, it being understood that only preferred embodiments have been shown and described. All changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein. This application former incorporates by reference the entire contents of U.S. provisional patent application No. 61/749,512, filed on Jan. 7, 2013, to which this application claims priority.

What is claimed is:

1. A protective bandage device comprising:
a. a top portion having
i. an inner and outer surface, said inner surface defining a hollow concave chamber between said top portion and the surface of the skin of a person on which the device is placed;
ii. a sloping wall portion; and
b. a bottom portion,
wherein said top and bottom portions reversibly couple to provide open and closed positions, wherein a hinge member joins the top and bottom portions allowing the top and bottom portions to remain attached when in the open position.

2. A bandage device as is claim 1 further comprising a comfort band.

3. A bandage device as in claim 2 wherein said comfort band interposes between the top and bottom portions covering said bottom portion, and contacting the skin of said person around a wound.

4. A bandage device as in claim 3 wherein the top portion of said device has at least one ventilation opening.

5. A bandage device as in claim 4 wherein said top portion comprises at least one ridge.

6. A bandage device as in claim 5 wherein said top portion further comprises a central aperture.

7. A bandage device as in claim 1 further comprising interacting coupling members on said top and bottom portions.

8. A bandage device as in claim 7 further comprising a tab member on said top portion.

9. A method of protecting a wound on a person in need thereof comprising applying to a patient in need thereof a device comprising
a. a top portion having
i. an inner and outer surface, said inner surface defining a hollow concave chamber between said top portion and the surface of the skin of a person on whom the device is placed;
ii. a sloping wall portion; and
b. a bottom portion,
wherein said top and bottom portions reversibly couple to provide open and closed positions, wherein a hinge member joins the top and bottom portions allowing the top and bottom portions to remain attached when in the open position.

10. A method as in claim 9 wherein said bandage device is applied to a wound substantially simultaneously with other treatments selected from the group consisting of conventional bandages, gauze pads, topical medicaments, antibiotics, anti-inflammatory agents, and pain reducers

11. A method as in claim 10 wherein said bandage device is applied on from day 1 to day 10 after said wound is sustained.

12. A protective bandage device as in claim 8 wherein said top portion further comprises a tube passage.
